SEQUENCE LISTING (2 sequences)

SEQ ID NO: 1 - DNA, 20 bp, Artificial Sequence
  Nectin-4 forward primer
caaaatctgtggcacattgg

SEQ ID NO: 2 - DNA, 20 bp, Artificial Sequence
  Nectin-4 reverse primer
gctgacatggcagacgtaga

(19) United States
(12) Patent Application Publication
(10) Pub. No.: US 2024/0352524 A1
(43) Pub. Date: Oct. 24, 2024

(54) PCR KIT FOR DIAGNOSING ASTHMA OR ASTHMA EXACERBATION AND METHOD FOR PROVIDING INFORMATION FOR DIAGNOSING ASTHMA OR ASTHMA EXACERBATION BY USING SAME

(71) Applicant: SOONCHUNHYANG UNIVERSITY INDUSTRY ACADEMY COOPERATION FOUNDATION, Asan-si Chung cheongnam-do (KR)

(72) Inventors: An Soo JANG, Bucheon-si, Gyeonggi-do (KR); Pu Reun Ha Neul LEE, Jangseong-gun, Jeollanam-do (KR)

(21) Appl. No.: 18/039,563

(22) PCT Filed: Nov. 26, 2021
(86) PCT No.: PCT/KR2021/017595

§ 371 Date: May 31, 2023

(30) Foreign Application Priority Data
Dec. 2, 2020 (KR) 10-2020-0166254

(51) Int. Cl. C12Q 1/6883; C12Q 1/6851

(52) U.S. Cl. CPC C12Q 1/6883; C12Q 1/6851; C12Q 2600/158

(57) ABSTRACT

An embodiment of the disclosure provides: a PCR kit for diagnosing asthma or asthma exacerbation, comprising an agent for measuring the mRNA level of a nectin-4 gene; and a method for providing information for diagnosing asthma or asthma exacerbation by using same. According to an embodiment of the disclosure, there is the effect of providing: a PCR kit for diagnosing asthma or asthma exacerbation, which enables the administration of an appropriate dose of a drug for asthma treatment by diagnosing not only asthma but also asthma exacerbation; and a method for providing information for diagnosing asthma or asthma exacerbation by using same.

### INCORPORATION OF SEQUENCE LISTING

This application contains a sequence listing submitted in Computer Readable Form (CRF). The CFR file containing the sequence listing is entitled “2-PJK4686508-SQListing.txt”, which was created and modified on Nov. 7, 2023, and is 902 bytes in size. The information in the sequence listing is incorporated herein by reference in its entirety.

### TECHNICAL FIELD

The disclosure relates to a PCR kit for diagnosing asthma or asthma exacerbation and a method for providing information for diagnosing asthma or asthma exacerbation using the same, and more particularly, to a PCR kit for diagnosing asthma or asthma exacerbation by measuring nectin-4 expression and a method for providing information for diagnosing asthma or asthma exacerbation using the same.

### BACKGROUND ART

Asthma refers to a disease in which the airway is repeatedly narrowed due to a chronic inflammatory reaction of the bronchi and symptoms such as coughing, wheezing (breathing with high-pitched whistling sound), and convulsive dyspnea occur repeatedly and convulsively. Asthma may be caused by diathesis of the individual or allergens such as house dust, mites, and pollen, but recently, patients with asthma exacerbated by yellow dust or fine dust are rapidly increasing.

Since asthma is a chronic and recurrent disease that is difficult to treat at once, it is very important to control symptoms well and normalize lung function to maintain normal daily life. In addition, even though early diagnosis of asthma and steady drug treatment are important because the condition can worsen if the symptoms are long, many patients often mistake asthma symptoms for a cold, so early diagnosis and treatment are difficult.

Further, in order to treat asthma, inhaled combination therapy has recently been used, which usually consists of corticosteroids and long-acting β-agonist or leukotriene modifiers (Mishra et al., 2013). However, as described above, asthma is difficult to be perfectly cured and drugs must continue to be administered for treatment. General drugs for asthma treatment have a lot of adverse effects such as growth suppression, eye problem, hypertension, hyperlipidemia, gastric ulcer, neurotoxicity, and so on (Wise 2014 Ciriaco et al., 2013).

Therefore, early diagnosis of asthma is important. In addition, it is required to develop a diagnostic kit for significantly diagnosing the degree of asthma exacerbation, thereby minimizing side effects and enabling continuous maintenance through drug administration in a prescribed amount.

### DOCUMENT OF RELATED ART

Japanese Patent Registration No. 3979660

### DISCLOSURE

### Technical Problem

The technical problem to be solved by the present disclosure is to provide a PCR kit capable of diagnosing asthma or asthma exacerbation using an agent capable of measuring a mRNA level of a nectin-4 gene.

In addition, the technical problem to be solved by the disclosure is to provide a method for providing information for diagnosing asthma or asthma exacerbation using an agent capable of measuring a mRNA level of a nectin-4 gene.

The technical problems to be solved by the present disclosure are not limited to the problems mentioned above, and other problems not mentioned may be clearly understood by those skilled in the art from the following descriptions.

### Technical Solution

In order to solve the above technical problems, one embodiment of the disclosure provides a PCR kit for diagnosing asthma or asthma exacerbation

A PCR kit for diagnosing asthma or asthma exacerbation according to one embodiment of the disclosure may comprise an agent for measuring a mRNA level of a nectin-4 gene; a DNA polymerase; deoxynucleotides (dNTPs); and a buffer.

The agent for measuring a mRNA level of a nectin-4 gene may comprise at least one selected from the group consisting of a primer pair, probe, LNA, and antisense nucleotide that specifically binds to the nectin-4 gene.

The agent for measuring a mRNA level of a nectin-4 gene may comprise a primer pair consisting of nucleotide sequences of SEQ ID NOs: 1 and 2.

The PCR kit may be an RT-PCR kit.

The RT-PCR kit may comprise a reverse transcriptase polymerization (RT-PCR) kit, a competitive reverse transcriptase polymerase reaction (competitive RT-PCR) kit, a real-time reverse transcriptase polymerase reaction (real time quantitative RT-PCR), and a quantitative RT-PCR kit.

In order to solve the above technical problems, another embodiment of the disclosure provides a method for providing information for diagnosing asthma.

According to one embodiment of the disclosure, the method for providing information for diagnosing asthma may comprise the steps of measuring an expression level of the nectin-4 gene in a sample isolated from a subject suspected of having asthma using the PCR kit for diagnosing asthma or asthma exacerbation according to one embodiment of the disclosure; comparing the expression level of the nectin-4 gene measured in the above step with the expression level of the nectin-4 gene of a normal subject; and determining the asthma if the expression level of the nectin-4 gene in the sample isolated from the subject suspected of having asthma is higher than the expression level of the nectin-4 gene of the normal subject.

In order to solve the above technical problems, still another embodiment of the disclosure provides a method for providing information for diagnosing asthma exacerbation.

According to one embodiment of the disclosure, the method for providing information for diagnosing asthma exacerbation may comprise the steps of measuring an expression level of the nectin-4 gene in a sample isolated from a subject suspected of having exacerbated asthma using the PCR kit for diagnosing asthma or asthma exacerbation according to one embodiment of the disclosure; comparing the expression level gene measured in the above step with the expression level of a subject from a stable asthma group; and determining the exacerbated asthma if the expression level of the nectin-4 gene in the sample isolated from the subject suspected of having exacerbated asthma is higher than the expression level of the nectin-4 gene of the subject of the stable asthma group.

### Advantageous Effects

According to one embodiment of the present disclosure, there is an effect capable of providing a PCR kit capable of diagnosing asthma or asthma exacerbation using an agent capable of measuring a mRNA level of a nectin-4 gene.

In addition, according to one embodiment of the present disclosure, there is an effect of capable of providing a method for providing information capable of diagnosing not only asthma diagnosis but also asthma exacerbation by using an agent capable of measuring a mRNA level of a nectin-4 gene.

The effects of the present disclosure are not limited to the above-mentioned effects, and it should be understood that the effects of the present disclosure include all effects that could be inferred from the configuration of the invention described in the detailed description of the invention or the appended claims.

### DESCRIPTION OF DRAWINGS

FIG. 1 is a flowchart schematically illustrating a method for providing information for diagnosing asthma according to one embodiment of the disclosure.

FIG. 2 is a flowchart schematically illustrating a method for providing information for diagnosing asthma exacerbation according to one embodiment of the disclosure.

FIG. 3 is a diagram illustrating an analysis of a nectin-4 expression at an RNA level in a normal control group, a stable asthma group and an exacerbated asthma group.

FIG. 4 is a graph illustrating a correlation between a nectin-4 level in plasma of asthmatic patients and clinical variables of FVC.

### MODE FOR INVENTION

Hereinafter, embodiments of the present disclosure will be explained with reference to the accompanying drawings. The invention, however, may be implemented in various different ways or forms, and should not be construed as limited to the embodiments set forth herein. Also, in order to clearly explain the embodiments of the present disclosure, portions that are not related to the invention are omitted, and like reference numerals are used to refer to like elements throughout.

Throughout the specification, it will be understood that when a portion is referred to as being “connected (accessed, contacted, coupled)” to another portion, it can be “directly connected to” the other portion, or “indirectly connected to” the other portion having intervening portions present. Also, when a component “includes” an element, unless there is another opposite description thereto, it should be understood that the component does not exclude another element but may further include another element.

Terms used herein may be merely to describe a certain embodiment, and may not be intended to limit the disclosure. The singular expressions may include plural expressions unless the context clearly dictates otherwise. It will be further understood that as used herein, the terms such as “comprise”, “include”, etc. denote the presence of stated features, numbers, steps, operations, components, parts, or combination thereof, but do not exclude the presence of or a possibility of addition of one or more other features, numbers, steps, operations, components, parts, or combination thereof.

Hereinafter, embodiments of the present disclosure will be described in detail with reference to the accompanying drawings.

A PCR kit for diagnosing asthma or asthma exacerbation according to one embodiment of the disclosure will be described.

A PCR kit for diagnosing asthma or asthma exacerbation according to an embodiment of the disclosure may comprise an agent for measuring a mRNA level of a nectin-4 gene; a DNA polymerase; deoxynucleotides (dNTPs); and a buffer.

The term “asthma” used in the disclosure may be bronchial asthma, allergic asthma, atopic asthma, non-atopic asthma, excise-induced asthma, aspirin-induced asthma, cardiac asthma, or alveolar asthma, but is not limited thereto, and it is a comprehensive disease name that collectively refers to various diseases characterized by the inflammatory response of the airways and the resulting damage and changes in airway tissues.

The “nectin-4” used in the disclosure is composed of a series of cell adhesion molecules that are involved in Ca²⁺-independent cell adhesion, and functions by binding to afadin, a protein that binds to actin. However, the biological significance or clinical potential of nectin-4 in asthma has not yet been researched.

The nectin-4 has a characteristic that the gene expression level or the expression level of the corresponding protein is changed in an individual with asthma compared to a normal control group, and more specifically, a characteristic of increased expression level, so that the nectin-4 may be used as an indicator for diagnosing asthma.

The term “mRNA level measurement” used in the disclosure refers to measuring an amount of mRNA by identifying the presence and expression level of mRNA of target genes in a target sample. In the disclosure, it refers to measuring the mRNA level of the nectin-4 gene for diagnosing asthma.

The agent for measuring the mRNA level of the nectin-4 gene may comprise at least one selected from the group consisting of a primer pair, probe, LNA, and antisense nucleotide that specifically binds to the above gene.

The term “primer pair” used in the disclosure refers to a set consisting of a forward primer and a reverse primer used to amplify a target gene through a polymerase chain reaction.

As used herein, the term “probe” refers to a labeled nucleic acid fragment or peptide capable of specifically binding to mRNA or protein. Specific examples may be ones that are prepared in the form of oligonucleotide probes, single stranded DNA probes, double stranded DNA probes, RNA probes, oligonucleotide peptide probes, polypeptide probes, and the like.

The term “LNA” used in the disclosure is a locked nucleic acid. This refers to one modified nucleic acid structure, which is formed by a methylene linkage between an oxygen atom attached to carbon 2 and carbon atom 4 of the pentose structure based on the structural form of ribose nucleic acid (RNA).

As used herein, the term “antisense nucleotide” refers to DNA or RNA or derivatives thereof containing a nucleic acid sequence complementary to a specific mRNA sequence, and the antisense nucleotide binds to a complementary sequence in mRNA and servers to inhibit translation of mRNA into protein. An antisense oligonucleotide sequence refers to a DNA or RNA sequence that is complementary to the mRNA of the genes and binds to the mRNA. This can inhibit translation of the mRNA of the gene, translocation into a cytoplasm, maturation, or any other vital activity for overall biological function. The length of the antisense oligonucleotide may be 6 to 100 bases, preferably 8 to 60 bases, more preferably 10 to 40 bases. The antisense oligonucleotide may be synthesized in vitro by a commonly employed method and administered in vivo, or the antisense oligonucleotide may be synthesized in vivo. One example of synthesizing the antisense oligonucleotides in vitro is using RNA polymerase I. One example of synthesizing the antisense RNA in vivo is to transcribe the antisense RNA using a vector with an origin of a multiple cloning site (MCS) in the opposite direction. Preferably, the antisense RNA has a translational stop codon in the sequence so that it is not translated into a peptide sequence.

Most preferably, the agent for measuring the mRNA level of the nectin-4 gene may comprise a primer pair consisting of the nucleotide sequences of SEQ ID NOs: 1 and 2.

Table 1 is a table showing the nucleotide sequences of the primer pairs.

TABLE 1

| | |
|---|---|
| SEQ ID NO: 1 | 5′-CAAAATCTGTGGCACATTGG-3′ |
| SEQ ID NO: 2 | 5′-GCTGACATGGCAGACGTAGA-3′ |

As described above, the agent for measuring the mRNA level of the nectin-4 gene of the disclosure comprises the primer pair having the above-described specific nucleotide sequence, so that the agent can diagnose asthma or asthma exacerbation by measuring the mRNA level of the nectin-4 gene.

The PCR kit may be an RT-PCR kit.

The term “RT-PCR” used in the disclosure comprises all of a reverse transcriptase polymerization (RT-PCR) kit, a competitive reverse transcriptase polymerase reaction (competitive RT-PCR) kit, a real-time reverse transcriptase polymerase reaction (real time quantitative RT-PCR), and a quantitative RT-PCR kit, and may comprise the primer pair consisting of the nucleotide sequences of SEQ ID NOs: 1 and 2. The primer pair is a nucleotide having a sequence specific to the nucleic acid sequence of the Nectin-4 gene.

The PCR kit may further comprise essential elements required for RT-PCR, such as a DNA polymerase, deoxynucleotides (dNTPs), a buffer, an appropriate tube for carrying out the polymerization reaction, and distilled water.

As used herein, the term “diagnosis” means identifying the presence or characteristics of a pathological condition. In this case, the diagnosis may comprise identifying not only the presence of the pathological condition, but also the progress or exacerbation of the pathological condition.

According to one embodiment of the disclosure, there is an effect of providing a PCR kit capable of diagnosing asthma or asthma exacerbation using the agent capable of measuring the mRNA level of the nectin-4 gene with the above characteristic configurations.

A method for providing information for diagnosing asthma according to another embodiment of the disclosure will be described.

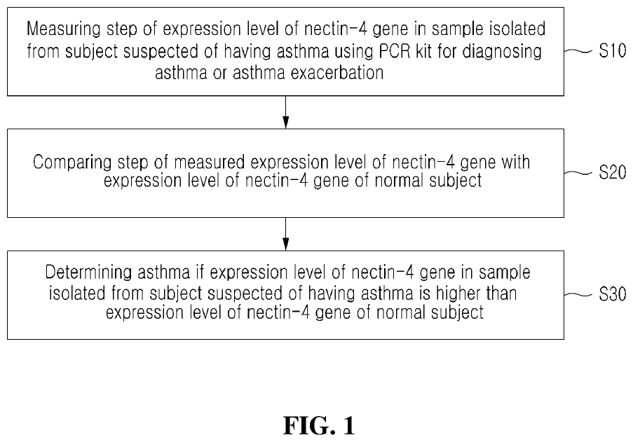

FIG. 1 is a flowchart schematically illustrating a method for providing information for diagnosing asthma according to one embodiment of the disclosure.

Referring to FIG. 1, a method for providing information for diagnosis of asthma according to one embodiment of the disclosure may comprise the steps of measuring an expression level of the nectin-4 gene in a sample isolated from a subject suspected of having asthma using the PCR kit for diagnosing asthma or asthma exacerbation according to one embodiment of the disclosure (S10); comparing the expression level of the nectin-4 gene measured in the above step with the expression level of the nectin-4 gene of a normal subject (S20); and determining asthma if the expression level of the nectin-4 gene in the sample isolated from the subject suspected of having asthma is higher than the expression level of the nectin-4 gene of the normal subject (S30).

In this case, the PCR kit for diagnosing asthma or asthma exacerbation may be according to one embodiment of the disclosure.

The term “sample” used in the disclosure comprises, but is not limited to, tissues, cells, whole blood, serum, plasma, saliva, cerebrospinal fluid or urine samples, which shows a difference in gene expression levels due to the onset of asthma.

The nectin-4 has a characteristic that the gene expression level or the expression level of the corresponding protein is changed in a subject with asthma compared to a normal control group, and more specifically, a characteristic of increased expression level. Therefore, if the expression level of the nectin-4 gene in the sample isolated from the subject suspected of asthma is higher than the expression level of the nectin-4 gene of the normal subject, it can be determined as asthma.

Here, the PCR kit, most preferably, may be a RT-PCR kit, but is not limited thereto.

Here, the PCR kit may comprise the primer pair consisting of the nucleotide sequences of SEQ ID NOs: 1 and 2, which are nucleotides having a sequence specific to the nucleic acid sequence of the nectin-4 gene, so that the expression level of the mRNA of the nectin-4 may be specifically measured.

Here, the RT-PCR kit may comprise all of a reverse transcriptase polymerase reaction (RT-PCR) kit, a competitive reverse transcriptase polymerase reaction (competitive RT-PCR) kit, a real time reverse transcriptase polymerase reaction (real time quantitative RT-PCR) kit, and a quantitative polymerase reaction (quantitative RT-PCR) kit, but is not limited thereto, and any known RT-PCR kit may be used without limitation.

Here, the PCR kit may further comprise essential elements required for RT-PCR, such as a DNA polymerase, deoxynucleotides (dNTPs), a buffer, an appropriate tube for carrying out the polymerization reaction, and distilled water.

A method for providing information for diagnosing asthma exacerbation according to another embodiment of the disclosure will be described.

Here, descriptions of components identical or similar to those of the above-described embodiment are replaced with those described in the above-described embodiment.

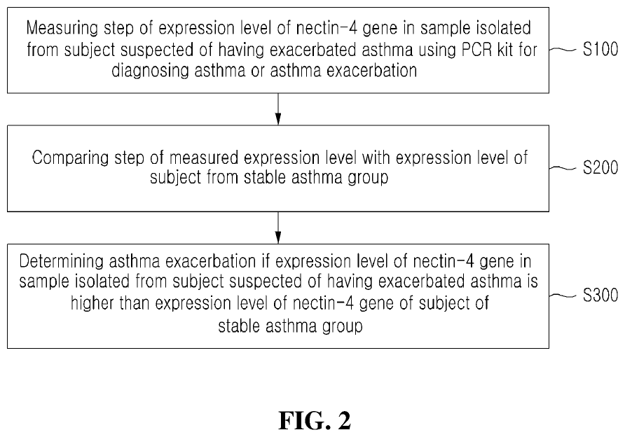

FIG. 2 is a flowchart schematically illustrating a method for providing information for diagnosing asthma exacerbation according to one embodiment of the disclosure.

Referring to FIG. 2, a method for providing information for diagnosing asthma exacerbation according to one embodiment of the disclosure may comprise the steps of measuring an expression level of the nectin-4 gene in a sample isolated from a subject suspected of having exacerbated asthma using the PCR kit for diagnosing asthma or asthma exacerbation according to one embodiment of the disclosure (S100); comparing the expression level gene measured in the above step with the expression level of a subject from a stable asthma group (S200); and determining exacerbated asthma if the expression level of the nectin-4 gene in the sample isolated from the subject suspected of having exacerbated asthma is higher than the expression level of the nectin-4 gene of the subject of the stable asthma group (S300).

Here, the PCR kit for diagnosing asthma or asthma exacerbation may be the PCR kit according to one embodiment of the disclosure.

Here, the PCR kit may comprise the primer pair consisting of the nucleotide sequences of SEQ ID NOs: 1 and 2, which are nucleotides having a sequence specific to the nucleic acid sequence of the nectin-4 gene, so that the expression level of the mRNA of the nectin-4 may be specifically measured.

As used herein, the term “stable asthma” refers to a patient group with stable symptoms who have asthma symptoms, but whose symptoms have not increased for at least the past 4 weeks or who do not require additional medication.

As used herein, the term “exacerbated asthma” refers to a sustained or rapid increase in asthma symptoms such as shortness of breath, wheezing, chest tightness, and decreased respiratory airflow.

Here, the nectin-4 not only has a characteristic of increased gene expression level or increased expression level of the corresponding protein in the subject with asthma compared to a normal control, but also shows a higher expression level in the exacerbated asthma group compared to that in the stable asthma group. Therefore, it can be determined as exacerbated asthma if the expression level of the nectin-4 gene in the sample isolated from the subject suspected of having exacerbated asthma is higher than the expression level of the nectin-4 gene in the subject from the stable asthma group.

According to one embodiment of the disclosure, there is an effect of providing a method for providing information for diagnosing asthma or asthma exacerbation using the agent capable of measuring the mRNA level of the nectin-4 gene due to the structural features as described above, so that not only asthma but also asthma exacerbation can be diagnosed so that an appropriate dose of a drug for asthma treatment can be administered.

Hereinafter, the disclosure will be described in more detail through experimental examples. However, the disclosure is not limited to the following experimental examples.

### <Experimental Example 1> Identification of Nectin-4 Expression at RNA Level in Normal Control Group, Stable Asthma Group and Exacerbated Asthma Group

Using the PCR kit comprising a pair of primers consisting of the nucleotide sequences of SEQ ID NOs: 1 and 2 according to the disclosure, an experiment for identifying the nectin-4 expression at the RNA level was identified in the blood of patients in the normal control group, stable asthma group, and exacerbated asthma group was conducted.

The experiment to identify the nectin-4 expression at the RNA level in the patient's blood comprises the steps of preparing an RNA sample from a specimen, amplifying the nectin-4 gene in the specimen sample by a real-time PCR using the prepared RNA sample, the nectin-4 primer pair, and a marker representing an amount of gene amplification, and then quantitatively measuring the amount of nectin-4 in the sample based on the amount of gene amplification.

Here, a SYBR green technique was used to detect the amplified DNA. In the SYBR Green technique, SYBR Green dye is inserted into the amplified DNA during the real-time PCR amplification process, binds to the double-stranded DNA synthesized by the PCR reaction, and generates a fluorescent signal. Thus, this is a method capable of measuring the presence or absence of a target gene and the amount of amplification products produced therefrom by detecting such a fluorescent signal. In addition, the real-time PCR technique using a dual labeled probe is a method using the dual labeled probe in which a fluorescent substance is labeled at the 5′ end and a quencher is labeled at the 3′ end. Thus, this is a method that can identify whether the double labeled probe has annealed with the PCR amplification product of the target gene through the fluorescent signal from the labeled probe, and can measure the presence or absence of the target gene and an amount of amplification product therefrom. It is apparent that in the disclosure, the real-time PCR known in the art, such as the TaqMan probe method, can be applied in addition to the above methods.

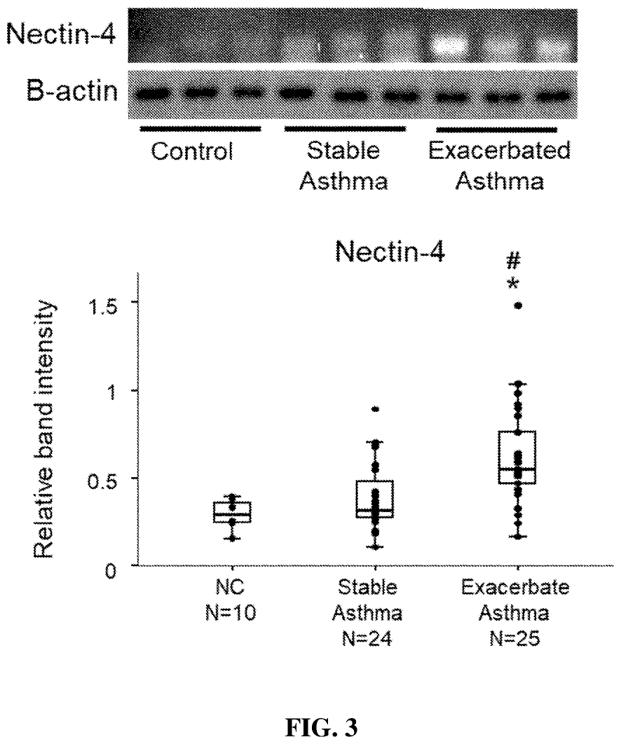

FIG. 3 is a diagram illustrating an analysis of a nectin-4 expression at an RNA level in a normal control group, a stable asthma group and an exacerbated asthma group.

Referring to FIG. 3, it can be confirmed that the expression of the nectin-4 is increased in the blood of asthma patients compared to that in the control group, and the mRNA expression level of nectin-4 is significantly higher in the exacerbated asthma group than that in the stable asthma group.

Through this, by using the PCR kit comprising the primer pair consisting of the nucleotide sequences of SEQ ID NOs: 1 and 2 according to one embodiment of the disclosure, asthma exacerbation as well as asthma can be diagnosed, thereby administering an appropriate dose of a drug for asthma treatment.

### <Experimental Example 2> Correlation Between Nectin-4 Level and FVC in Asthma Patients

An experiment was conducted to determine the correlation between the nectin-4 level in asthma patients and forced vital capacity (FVC).

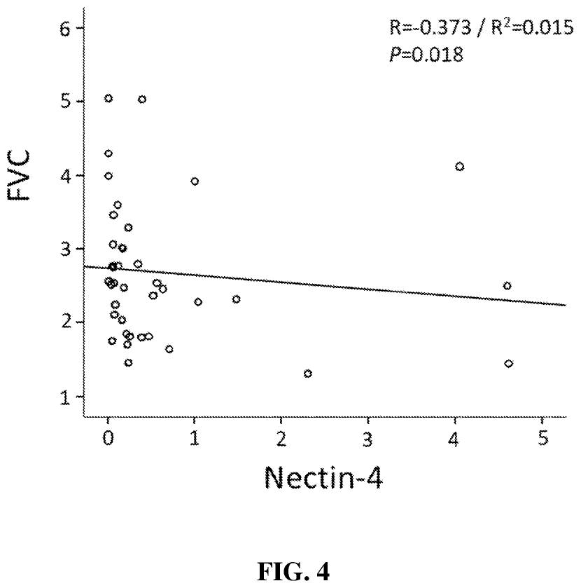

FIG. 4 is a graph illustrating a correlation between a nectin-4 level in plasma of asthmatic patients and clinical variables of FVC.

Referring to FIG. 4, the nectin-4 level in the plasma of asthmatic patients has correlation with FVC % pred. (R=−0.0373, P=0.018).

The above description of the present disclosure is used for illustration and those skilled in the art will understand that the present disclosure can be easily modified to other detailed forms without changing the technical spirit or an essential feature thereof. Therefore, it is to be understood that the aforementioned embodiments are illustrative in all aspects and not restrictive. For example, each component described as a single type may be implemented to be distributed and similarly, components described to be distributed may also be implemented in a combined form.

The scope of the present disclosure is represented by the claims to be described below, and it is to be interpreted that the meaning and scope of the claims and all the changes or modified forms derived from the equivalents thereof come within the scope of the present disclosure.

1. A PCR kit for diagnosing asthma or asthma exacerbation, comprising:
an agent for measuring a mRNA level of a nectin-4 gene;
a DNA polymerase;
deoxynucleotides (dNTPs); and
a buffer.

2. The PCR kit of claim 1, wherein the agent for measuring the mRNA level of the nectin-4 gene comprises at least one selected from the group consisting of a primer pair, probe, LNA, and antisense nucleotide that specifically binds to the nectin-4 gene.

3. The PCR kit of claim 1, wherein the agent for measuring the mRNA level of the nectin-4 gene comprises a primer pair consisting of nucleotide sequences of SEQ ID NOs: 1 and 2.

4. The PCR kit of claim 1, wherein the PCR kit is an RT-PCR kit.

5. The PCR kit of claim 4, wherein the RT-PCR kit comprises a reverse transcriptase polymerization (RT-PCR) kit, a competitive reverse transcriptase polymerase reaction (competitive RT-PCR) kit, a real-time reverse transcriptase polymerase reaction (real time quantitative RT-PCR), and a quantitative RT-PCR kit.

6. A method for providing information for diagnosing asthma comprising:
measuring an expression level of a nectin-4 gene in a sample isolated from a subject suspected of having asthma using the PCR kit for diagnosing asthma or asthma exacerbation of claim 1;
comparing the expression level of the nectin-4 gene in the sample with an expression level of a nectin-4 gene of a normal subject; and
determining the asthma if the expression level of the nectin-4 gene in the sample isolated from the subject suspected of having asthma is higher than the expression level of the nectin-4 gene of the normal subject.

7. A method for providing information for diagnosing asthma exacerbation comprising:
measuring an expression level of a nectin-4 gene in a sample isolated from a subject suspected of having exacerbated asthma using the PCR kit for diagnosing asthma or asthma exacerbation of claim 1;
comparing the expression level of the nectin-4 gene in the sample with an expression level of a nectin-4 gene of a subject from a stable asthma group; and
determining the exacerbated asthma if the expression level of the nectin-4 gene in the sample isolated from the subject suspected of having exacerbated asthma is higher than the expression level of the nectin-4 gene of the subject from the stable asthma group.
